(19) United States
(12) Patent Application Publication
(10) Pub. No.: US 2005/0027280 A1
(43) Pub. Date: Feb. 3, 2005

(54) Hook and loop disposable undergarment

(76) Inventors: La'Tanya Y. Patty-Brown, Memphis, TN (US); William C. Brown, Memphis, TN (US)

Correspondence Address: LITMAN LAW OFFICES, LTD., P.O. BOX 15035 CRYSTAL CITY STATION, ARLINGTON, VA, 22215, US

(21) Appl. No.: 10/627,781

(22) Filed: Jul. 28, 2003

(51) Int. Cl. A61F 13/15

(52) U.S. Cl. 604/392

(57) ABSTRACT

A hook and loop disposable undergarment for use by toilet training toddlers and incontinent adults. The hook and loop disposable undergarment has a generally rectangular flap portion, a belt portion and elastic supports. The generally rectangular flap portion can be folded back and releasably secured from the back of the belt portion, allowing the user to sit on a toilet and go to the bathroom without taking the hook and loop disposable undergarment off or pulling it down.

### BACKGROUND OF THE INVENTION

1. Field of the Invention

The present invention relates to a hook and loop disposable undergarment for toddlers and incontinent adults.

2. Description of the Related Art

Disposable undergarments are often used by toddlers and incontinent adults. Toddlers use disposable undergarments as part of their toilet training and incontinent adults use these disposable garments to protect themselves from the embarrassment from sudden incontinence. These types of disposable undergarments are reflected in the related art.

U.S. patent application Publication No. 2002/0099353 published on Jul. 25, 2002, outlines the use of a disposable absorbent article, that has at least one first fastening component disposed in a first waist region, and at least one second fastening component disposed in an opposite second waist region. The fastening components are adapted to releasably engage one another, and at least one of the fastening components make-up primary and secondary regions that have different releasable engagement properties.

U.S. patent application Publication No. 2002/0111596 published on Aug. 15, 2002, outlines the use of a pant-like absorbent garment having removable side panels, that are releasable and refastenable. The resulting garment can be opened and closed on either the front side of the garment, or the back side of the garment, and on either the left side or the right side of the garment. Furthermore, the removable side panels can be reused with a disposable chassis portion of the garment.

U.S. patent application Publication No. 2002/0138065 published on Sep. 26, 2002, outlines the use of a garment with a waistband with a crotch component, that can be permanently attached to the back of the waistband and be releasably attached to the front of the waistband. The crotch component fits between the user's legs during use, and the waistband wraps around the user's waist. The garment may be made of one, two or three-piece construction, and also has an absorbent core disposed generally in the crotch region.

U.S. patent application Publication No. 2002/0151858 published on Oct. 17, 2002, outlines the use of a disposable absorbent article to fit various torso sizes. The article has a laterally extending belt, which may be partly stretchable or elastic, and an insert member disposed vertically relative to the belt. The insert member has a proximal end for attachment to the belt, and a distal end having two spaced apart tab fasteners.

U.S. patent application Publication No. 2002/0188268 published on Dec. 12, 2002, outlines the use of an absorbent article that has elastomeric ear panels and a fastening system, that provides the user with different options as to how the absorbent article may be fitted to and removed from the wearer. The absorbent articles allow the wearer to choose between conventional and pull-on diaper configurations, or combinations thereof, and properly and comfortably fit a large range of wearer sizes.

U.S. Pat. No. 4,145,763 issued to Abrams et al., outlines the use of an undergarment for use in surgical, medical or other medical treatments. The undergarment is made up of at least two completely separable cloth panels, the panels being separable one from the other, and the separation and fastening being effected by the use of hook and loop fasteners or similar adhering materials. The undergarment may also contain a slit positioned near the urinary tract for a catheter or drain, for keeping a folded-back upper or lower side or crotch portion of the undergarment open and secured in place, during examination, treatment or surgery.

U.S. Pat. No. 4,610,680 issued to LaFleur, outlines the use of a disposable training panty. The panty is made up of a panty member adapted to form an enclosed waist opening and enclosed leg openings, from a panel having an inner layer of absorbent material and an outer layer of moisture impervious material. Preferably the openings are peripherally defined at least in part by elastic material.

U.S. Pat. No. 5,087,253 issued to Cooper, outlines the use of an incontinence garment, in the form of a universal combination diaper/training pant for children and adults. The combination may be made in a one-piece configuration, which would be disposable and may also be made in a two-piece configuration, which would be washable. An outer water-resistant portion is formed in the shape of a pair of training pants, and has an inner water-absorbing portion attached or positioned on the outer portion. An inner padding is also positioned between the outer water-resistant portions and the inner water-absorbing portion.

U.S. Pat. No. 5,100,399 issued to Janson et al., outlines the use of a baby's diaper, with a shaped support member of cloth having a modified I-shape, with a top end portion having lateral extensions, and a bottom end having shorter and slightly wider lateral extensions. There are rectangular strips of hook and loop material attached to the outer face of the support member, for engagement by strips of hook and loop material attached to the inner face of the support member.

U.S. Pat. No. 5,370,634 issued to Ando et al., outlines the use of an underpants type of disposable diaper. The disposable diaper is made up of a liquid permeable top sheet, a liquid impermeable back sheet, an absorbent member disposed in-between, and an elastic expandable member. The expandable member forms a gather to a pair of leg portions and front and rear waist body portions formed around the absorbent member. The front and rear waist body portions are connected and fixed together at both sides, to form a waist opening portion and a pair of leg opening portions.

U.S. Pat. No. 5,545,158 issued to Jessup, outlines the use of a disposable absorbent garment, made up of a pair of elastic side sections, front and back sections, and a continuous, selectively elasticized waistband operatively joined thereto. The waistband does not substantially inhibit the elasticity of the elastic side sections, and provides the same or different elasticity to the front and back sections.

Although each of the patent applications and patents outline useful inventions and devices, what is really needed is a diaper that can easily allow a toddler or an incontinent adult user to sit on a toilet and go to the bathroom while wearing the diaper. Such a diaper would be in great demand and be very successful in the marketplace.

None of the above inventions and patents, taken either singly or in combination, is seen to describe the instant invention as claimed. Thus a disposable garment for toilet training solving the aforementioned problems is desired.

### SUMMARY OF THE INVENTION

The invention is a hook and loop disposable undergarment for use by toilet training toddlers and incontinent adults. The hook and loop disposable undergarment has a generally rectangular flap portion, a belt portion and elastic supports. The generally rectangular flap portion can be folded back and releasably secured from the back of the belt portion, allowing the user to sit on a toilet and go to the bathroom without taking the hook and loop disposable undergarment off.

Accordingly, it is a principal object of the invention to provide a hook and loop disposable undergarment for toddlers and incontinent adults.

It is another object of the invention to provide a hook and loop disposable undergarment that can be worn while a user is sitting on a toilet and going to the bathroom.

It is a further object of the invention to provide a hook and loop undergarment that can be folded backwards and held in back of a user, while sitting on a toilet and going to the bathroom.

It is an object of the invention to provide improved elements and arrangements thereof for the purposes described which is inexpensive, dependable and fully effective in accomplishing its intended purposes.

These and other objects of the present invention will become readily apparent upon further review of the following specification and drawings.

### BRIEF DESCRIPTION OF THE DRAWINGS

FIG. 1 is an environmental, perspective view of a hook and loop disposable undergarment, according to the present invention.

FIG. 2 is a front perspective view of the hook and loop disposable undergarment.

FIG. 3 is a rear perspective view of an open hook and loop disposable undergarment.

Similar reference characters denote corresponding features consistently throughout the attached drawings.

### DETAILED DESCRIPTION OF THE PREFERRED EMBODIMENT

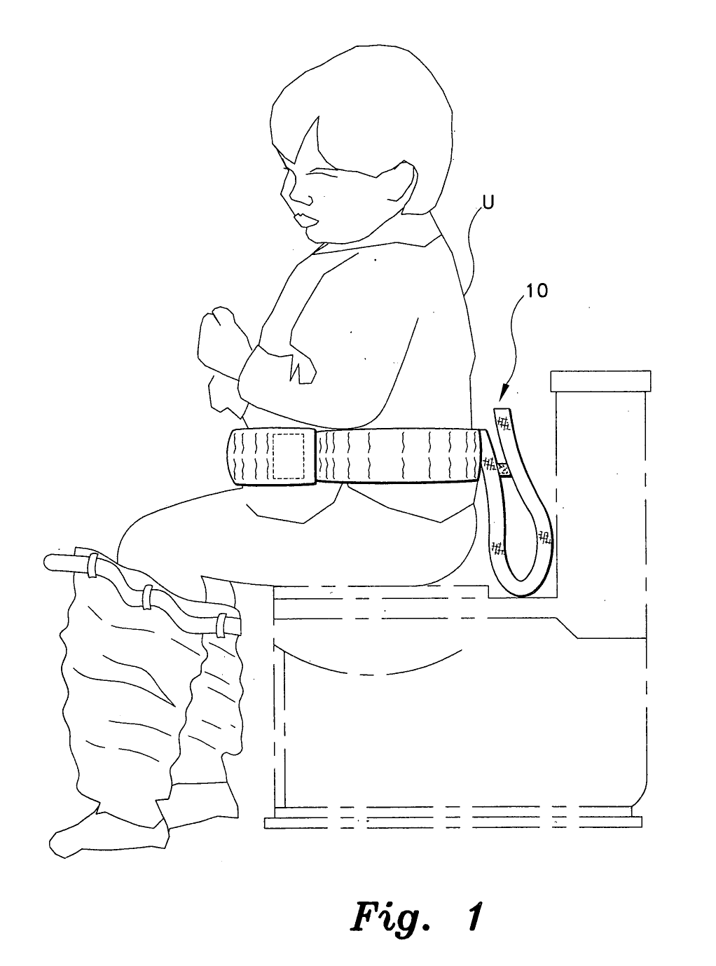

The present invention is a disposable undergarment 10 for a user U with a middle buttocks area, two legs, a midriff and a crotch area, as is depicted in FIG. 1.

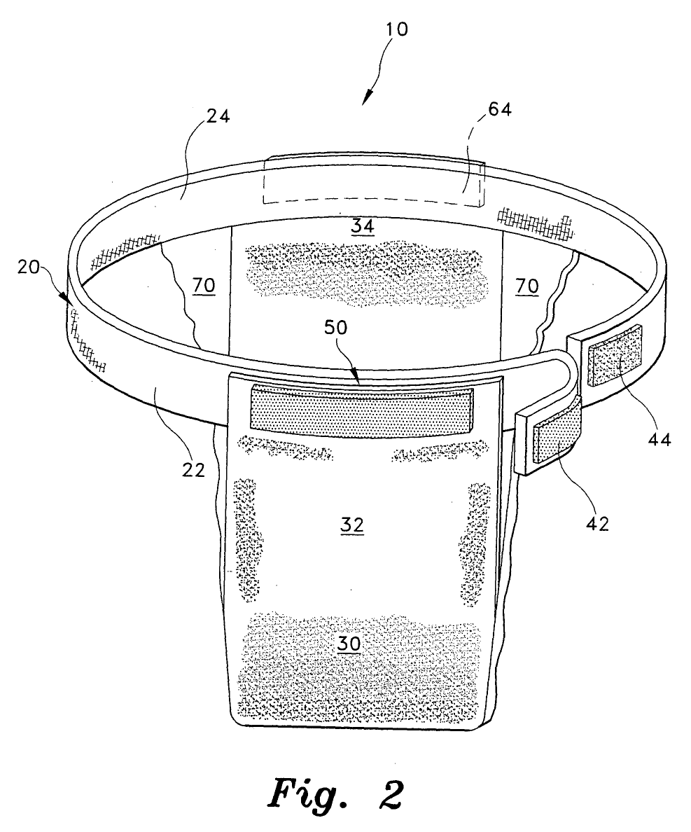

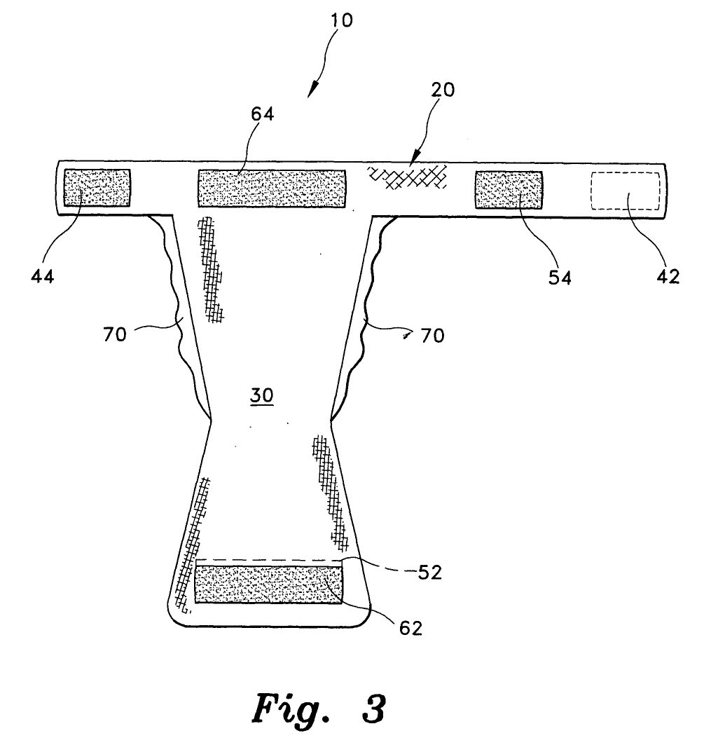

As are shown in FIG. 2 and FIG. 3, the disposable undergarment 10 comprises a belt portion 20 with a front 22 and a back 24, that is wrapped around the midriff of the user U and is releasably secured to hold the disposable undergarment 10 onto the user U. The disposable undergarment 10 further comprises a generally rectangular flap portion 30 with a front end 32 and a back end 34, with the back end 34 permanently secured to the back of the belt portion 24. The generally rectangular flap portion 30 covers the middle buttocks area of the user U and is wrapped between the two legs of the user U. The front end 32 of the generally rectangular flap portion 30 is then pulled up from between the two legs of the user U and over the crotch area of the user U, to the front of the belt portion 22 to be releasably secured. The front end 32 of the generally rectangular flap portion 30, can also be folded back to the back of the belt portion 24 and can be releasably secured, while the user U sits and uses a toilet.

The disposable undergarment 10 and its belt portion 20 are releasably secured around the midriff of the user U, with a first hook and loop fastener 40. The first hook and loop fastener 40 has two parts, a hook part 42 and a loop part 44. The first hook and loop fastener 40 used is VELCRO™, which is well-known to those schooled in the related art. The belt portion 20 comes in a variety of sizes to accommodate toddlers and incontinent adults, and the first hook and loop fastener 40 can be easily manually attached together or separated and detached.

The disposable undergarment 10 also has the front end 32 of the generally rectangular flap portion 30 releasably secured with a second hook and loop fastener 50 to the front of the belt portion 22. The second hook and loop fastener 50 has two parts, the hook part 52 and the loop part 54. The second hook and loop fastener 50 used is also VELCRO™, which is well-known to those schooled in the related art. The second hook and loop fastener 50 can also be easily manually attached together or separated and detached.

The disposable undergarment 10 can also have the front end 32 of the generally rectangular flap portion 30 folded back to the back of the belt portion 24, being releasably secured with a third hook and loop fastener 60. The third hook and loop fastener 60 also has two parts, a hook part 62 and a loop part 64. The third hook and loop fastener 60 used is also VELCRO™, which is well-known to those schooled in the related art. The third hook and loop fastener 60 can also be easily manually attached together or separated and detached. This is also a novel feature of the disposable undergarment 10, which allows a user U to sit on a toilet and easily go to the bathroom, without removing the disposable undergarment 10. The disposable undergarment 10 also utilizes elastic supports 70, which are provided between the back of the belt portion 24 and the back end 34 of the generally rectangular flap portion 30.

Use of the disposable undergarment 10 is uncomplicated. The disposable undergarment 10 can be used to potty train toddlers without removing the disposable garment 10. The disposable garment 10 is also ideal for incontinent adults, who must have immediate incontinence protection. The disposable garment 10 is also an attractive alternative to the popular “pull-up” training pants. The belt portion 20 is made of comfortable and elastic material and the generally rectangular flap portion 30 is made of absorbent material, to absorb any moisture from the user U.

It is to be understood that the present invention is not limited to the embodiment described above, but encompasses any and all embodiments within the scope of the following claims.

1. A disposable undergarment for a user with a middle buttocks area, two legs, a midriff and a crotch area, comprising:
a belt portion with a front and a back, that is wrapped around the midriff of the user and is releasably secured to hold the disposable undergarment onto the user; and
a generally rectangular flap portion with a front end and a back end, with the back end permanently secured to the back of the belt portion, said generally rectangular flap portion covering the middle buttocks area of the user and is wrapped between the two legs of the user, with the front end of the generally rectangular flap portion being pulled up from between the two legs of the user and over the crotch area of the user to the front of the belt portion to be releasably secured, said front end of the generally rectangular flap portion can also be folded back to the back of the belt portion and can be releasably secured, while the user sits and uses a toilet.

2. The disposable undergarment according to claim 1, wherein the belt portion is releasably secured around the midriff of the user, with a first hook and loop fastener.

3. The disposable undergarment according to claim 1, wherein the front end of the generally rectangular flap portion is releasably secured with a second hook and loop fastener to the front of the belt portion.

4. The disposable undergarment according to claim 1, wherein the front end of the generally rectangular flap portion is folded back to the back of the belt portion, being releasably secured with a third hook and loop fastener.

5. The disposable undergarment according to claim 1, wherein elastic supports are provided between the back of the belt portion and the back end of the generally rectangular flap portion.
